(19) United States
(12) Patent Application Publication
(10) Pub. No.: US 2026/0294742 A1
(43) Pub. Date: Oct. 1, 2026

(54) NOVEL 3D PRINTING CONCEPT FOR PHARMACEUTICAL DOSAGE FORMS VIA SELECTIVE LASER SINTERING

(71) Applicant: Merck Patent GmbH, Darmstadt (DE)

(72) Inventors: Thomas Kipping, Darmstadt (DE); Jonas Lindh, Uppsala (SE)

(21) Appl. No.: 19/476,797

(22) PCT Filed: May 21, 2024
(86) PCT No.: PCT/EP2024/063953

§ 371 Date: Oct. 21, 2025

(30) Foreign Application Priority Data
May 23, 2023 (EP) 23174835.1

(51) Int. Cl. A61J 3/10; A61K 9/20; A61K 31/405; B33Y 10/00; B33Y 80/00

(52) U.S. Cl. CPC A61J 3/10; A61K 9/2009; A61K 9/2027; A61K 9/2086; A61K 31/405; B33Y 10/00; B33Y 80/00

(57) ABSTRACT

The present invention relates to a process for producing a Pharmaceutical dosage form by powder bed fusion selective laser 3-dimensional printing, in particular selective laser sintering 3-dimensional printing (SLS), and a pharmaceutical dosage form manufactured by that process.

### RELATED APPLICATIONS

The present application is a National Stage Entry under 35 U.S.C. § 371 of International Application No. PCT/EP2024/063953, filed May 21, 2024, which claims the benefit of priority of European Application. No. 23174835.1, filed May 23, 2023, the contents of each of which are incorporated herein by reference in their entireties.

### TECHNICAL FIELD

The present invention relates to a process for producing a pharmaceutical dosage form by powder bed fusion selective laser 3-dimensional printing, in particular selective laser sintering 3-dimensional printing (SLS), and a pharmaceutical dosage form manufactured by that process.

### BACKGROUND

Selective Laser Sintering (SLS) is an additive manufacturing process that creates a three-dimensional (3D) object layer-by-layer. The process applies layers of powder material on top of each other sequentially, where each layer of powder is sintered or coalesced together with a laser according to the computer aided drawing (CAD) geometry of the part.

SLS is a powder bed based additive manufacturing technique to produce complex three-dimensional parts. In SLS, a rasterized laser is used to scan over a bed of polymer powder, sintering it to form solid shapes in a layer-wise fashion. When the laser beam scans the powder, the powder melts due to the rising temperature, and layer by layer, the final part approaches full density and should result in properties of the bulk material (the polymer). By controlling the energy input it is possible to control the density of the sintered material and to achieve parts ranging from highly porous to almost fully dense.

Selective laser sintering (SLS) is a subset of powder bed fusion 3D printing which uses a laser beam to create solid objects by heating powder particles, fusing them together at their surfaces. Currently, the majority of commercially available SLS printers employ carbon dioxide (CO₂) lasers, which provide higher power at lower cost, permitting the use of a wide array of powdered thermoplastic materials. As such, applications of SLS span many fields, including the aerospace, automotive, military, medical, dentistry, engineering and electronics industries. In the pharmaceutical sector, therapeutic products can be fabricated using SLS printing if the feedstock material is a powder blend of a drug and thermoplastic polymer. This means that, compared with other 3D printing technologies, the feedstock material of SLS printing has the closest resemblance to that of traditional tableting. As such, it has been anticipated that SLS is more amenable for pharmaceutical use. Whilst other 3D printing technologies, such as binder jetting, are also based on powdered materials, being a solvent-free process makes SLS a faster process, wherein the need for additional drying steps to evaporate any residual binder is avoided.

Currently described 3D printing concepts for selective laser sintering to create pharmaceutical dosage forms are utilizing a layer by layer approach where a pre-mixed powder is used which usually consists of at least one polymer, an API and potentially a certain absorber material depending on the light/laser source (Awad et al., 2020; International Journal of Pharmaceutics 586:119594). This concept requires a pre-mix of all components to achieve a homogenous sintering.

The need of a pre-mix of API and polymer makes the formulation development more complex, as for each printing step individual mixture need to be prepared. Also de-mixing effects can occur during the process. The use of a defined pre-mix limits dedicated dose adaptation and flexibility during formulations development.

Therefore, there is a need for a process for producing a pharmaceutical dosage form by powder bed fusion selective laser 3-dimensional printing, wherein the drug content can be individually adjusted within a printing step. Furthermore, there is a need for such a process, wherein the de-mixing effects during the process are avoided. Additionally, such a process should desirably yield to pharmaceutical dosage forms with an improved structure consistency and/or a higher drug loading compared to the conventional processes.

### SUMMARY OF THE INVENTION

It was surprisingly found that a process for producing a pharmaceutical dosage form by powder bed fusion selective laser 3-dimensional printing, comprising the steps of (a) providing a first sinter powder, essentially consisting of an active pharmaceutical ingredient or essentially consisting of a mixture of at least two active pharmaceutical ingredients, in a first reservoir platform, (b) providing a second sinter powder, comprising an excipient, wherein said excipient absorbs electromagnetic radiation at a wavelength emitted by the laser, in a second reservoir platform, and (c) operating a selective laser sintering apparatus that fuses layers of the first and second sinter powder to produce the pharmaceutical dosage form, leads to an improved manufacturing process and pharmaceutical dosage forms with improved properties.

In a preferred embodiment of the invention, the powder bed fusion selective laser 3-dimensional printing comprises selective laser sintering 3-dimensional printing, selective laser melting 3-dimensional printing, electron beam melting 3-dimensional printing or multijet fusion or a mixture thereof, preferably selective laser sintering 3-dimensional printing, preferably selective laser sintering 3-dimensional printing.

In a further preferred embodiment of the invention, the excipient is a polymer, in particular a polymer selected from the group consisting of acrylic-derived polymers, cellulose-derived polymers and polyvinyl-derived polymers and mixtures thereof.

In another aspect, the invention provides a pharmaceutical dosage form produced by the process as described above.

### DETAILED DESCRIPTION OF THE INVENTION

An embodiment of the invention is a process for producing a pharmaceutical dosage form by powder bed fusion selective laser 3-dimensional printing, comprising the steps of
(a) providing a first sinter powder, essentially consisting of an active pharmaceutical ingredient or essentially consisting of a mixture of at least two active pharmaceutical ingredients, in a first reservoir platform,
(b) providing a second sinter powder, comprising an excipient, wherein said excipient absorbs electromagnetic radiation at a wavelength emitted by the laser, in a second reservoir platform, and
(c) operating a selective laser sintering apparatus that fuses layers of the first and second sinter powder to produce the pharmaceutical dosage form.

In the conventional method of powder bed fusion selective laser 3-dimensional printing, in particular selective laser sintering 3-dimensional printing, for pharmaceutical applications, the one or more reservoirs are filled with a mixture of the active pharmaceutical ingredient (API) and an excipient.

According to the invention the powder bed fusion selective laser 3-dimensional printing comprises two reservoirs, whereas a first reservoir is filled with a sinter powder essentially consisting of an API. Provided API and excipient are utilized in separate chambers, no pre-mix of components is required. API content and release rates can be individually adjusted by selecting the targeted compartment. Variations can be performed by the amount of individual layers or even by variations of layer highs. Amount of individual drug or polymer layer can be adapted. The new system may allow the use of dedicated cartridge systems which can be directly supplied to the individual compartment chambers.

Furthermore, it was surprisingly found that the process according to the invention leads to pharmaceutical dosage forms with an improved homogeneity and/or structure consistency. In addition a higher drug loading of the resulting pharmaceutical dosage forms could be achieved.

The advantages of the inventive process as well as their improved characteristics of the pharmaceutical dosage forms are important aspects in the manufacture of pharmaceutical products.

According to the invention, the term “essentially consisting of an active pharmaceutical ingredient” means that the first sinter powder consists of the API or the first sinter powder predominantly comprises the API and at least one pharmaceutically acceptable additive. Preferably the pharmaceutically acceptable additive is a flow control agent. Flow control agents, such as silicon dioxide, are required for APIs that do not have a suitable flowablity in order to secure an adequate API flow and API content uniformity of the pharmaceutical dosage form.

In one embodiment, the first sinter powder consists of an active pharmaceutical ingredient or consists of a mixture of at least two active pharmaceutical ingredients.

In a further embodiment, the first sinter powder consists of from 95 to 100 percent by weight of at least one API and from 5 to 0 percent by weight of a pharmaceutically acceptable additive. In a further embodiment, the first sinter powder consists of from 97 to 100 percent by weight of at least one API and from 3 to 0 percent by weight of a pharmaceutically acceptable additive.

In a further embodiment, the first sinter powder consists of from 95 to 100 percent by weight of at least one API and from 5 to 0 percent by weight of a flow control agent. In a further embodiment, the first sinter powder consists of from 97 to 100 percent by weight of at least one API and from 3 to 0 percent by weight of a flow control agent.

In a further embodiment, the first sinter powder comprises at least one API but does not comprise an excipient as defined below. In a further preferred embodiment, the first sinter powder comprises at least one API but does not comprise an excipient as defined below that absorbs electromagnetic radiation at a wavelength emitted by the laser. In a further preferred embodiment, the first sinter powder comprises at least one API but does not comprise an excipient and a pharmaceutically acceptable additive as defined below that absorbs electromagnetic radiation at a wavelength emitted by the laser.

According to the invention, the term “powder bed fusion selective laser 3-dimensional printing” denotes a subset of four technologies: selective laser sintering (SLS), selective laser melting (SLM), electron beam melting (EBM) and multijet fusion (MJF). The technologies differ by the type of materials they employ and by the type and amount of light or radiation utilised to transmit energy to the powder bed. In all cases, objects are built layer-by-layer through the use of thermal energy resulting from the combination of increased temperature and the use of a light source and all use powders as their feedstock materials. One immediate benefit of this is that it permits fabrication of overhanging and/or intricate structures, without the need for a secondary support material, because the loose powder particles inside the bed act as a support, maintaining the integrity of the object during printing.

Thermoplastic polymers are used as the main feedstock material in SLS printing. The laser beam melts the surface of the powder particles, fusing them together, a process termed ‘sintering’. Because a relatively low-power laser is used, the printer itself heats the feedstock powder, so the laser needs only to provide a small increase in surface temperature of the powder to induce sintering. When the feed materials are metals or alloyed powders, the technology is normally called SLM or direct metal laser sintering (DMLS). EBM also uses metal and alloyed powders as its main feed material, although the energy required to sinter the particles is provided with an electron beam instead of a laser beam. The high intensity of the electron beam renders the powdered materials completely melted during the printing process.

MJF utilizes only one feedstock, nylon (for instance, PA 12), and it employs an infrared (IR) lamp as the energy source. Two additional components are needed in MJF: (i) a fusion agent, which is precisely deposited by an ink-jet head onto the printing regions, and (ii) a detailing agent, which is responsible for absorbing heat from the edges of the object. As such, only the regions coated with the fusion agent will melt, enhancing the printing efficiency and speed. The addition of the detailing agent decreases thermal bleeding (e.g. the spreading of heat across neighbouring regions) and enhances the printing resolution and accuracy.

In a preferred embodiment, the powder bed fusion selective laser 3-dimensional printing comprises selective laser sintering 3-dimensional printing, selective laser melting 3-dimensional printing, electron beam melting 3-dimensional printing or multijet fusion or a mixture thereof, preferably selective laser sintering 3-dimensional printing, more preferably, the powder bed fusion selective laser 3-dimensional printing is selective laser sintering 3-dimensional printing (SLS).

According to the invention, the term “selective laser sintering”, “SLS”, “selective laser sintering 3D printing” or “SLS 3DP” is a process in which a laser beam is used to sinter a powder bed filled with a sinter powder by scanning the laser according to the cross-section of a digital model. A version of the digital model is produced in a layer-by-layer fashion by laser scanning successive layers of powder.

The process will require a selective laser sintering printer equipped with a laser source and a galvanometric system for scanning the laser on the powder bed surface or, alternatively, a xy motion system where the actual laser source is moved to scan the powder bed or a mirror reflecting the laser source to achieve a xy motion of the laser spot within the powder bed. The printer must also provide, two reservoir platforms, a first reservoir platform with a first sinter powder and a second reservoir platform with a second sinter powder, a building platform and a powder application system to spread the powder in layers on the building platform (Spreader) as well as some heating capabilities to heat the build chamber and the surface of the powder bed.

Parameters that can be varied in SLS typically include the type of laser and thus its wavelength, as well as the laser power, scan speed, print resolution (layer height), beam spot size, surface temperature, chamber temperature, and the initial position of the build platform and its lowering speed. Also the powder dispenser may vary and the design of the scraper or other toolings, like a roller device. A laser is a device that emits light through a process of optical amplification based on the stimulated emission of electromagnetic radiation. The term “laser” originated as an acronym for “light amplification by stimulated emission of radiation”. Lasers emit light coherently.

Types of lasers used in SLS include, for example, CO₂ lasers, infrared lasers, and diode lasers such as blue diode lasers. In a preferred embodiment, the laser is a CO₂ laser.

The wavelength of electromagnetic radiation emitted by lasers suitable for SLS is typically within the range of 200 nm to 11 μm, typically in the near ultra-violet through to the mid-infrared part of the electromagnetic spectrum. For example, suitable lasers may emit electromagnetic radiation in the range of 315 nm to 1.4 μm, such as 400-610 nm, preferably 400-500 nm, more preferably 430-470 nm. Another suitable laser emits electromagnetic radiation in the range of 9.4 to 11 μm, such as 10.2-10.8 μm, preferably around 10.6 μm. Another suitable laser emits electromagnetic radiation in the range of 750-850 nm, such as approximately 800 nm.

The power of a laser is measured in Watts. This is referring to the optical power output of the laser beam, which is the continuous power output of continuous wave (CW) lasers, or the average power of a pulsed or modulated laser. Typically, SLS 3D printers use continuous wave lasers. Lasers suitable for SLS according to this invention typically have a power in the range of 0.5 W to 140 W. In all aspects and embodiments of this invention it is preferred if the laser has a power in the range of at least 1 W to less than 80 W, for example, 1.5-75 W. More preferably the laser power may be in the range of 1-30 W, such as 2-20 W, for example 3-15 W.

Suitable SLS 3D printers may use more than one laser. For example, printers may use two lasers, or more than two lasers.

The scan speed for SLS is the rate at which the laser moves over the powder bed. Suitable scan speeds for the current invention range from about 5 mm/s to about 50000 mm/s. The scan speed correlates directly with the laser beam interaction time. A slower scan speed results in a longer laser beam interaction time. In a preferred embodiment, applicable to all aspects of the invention, the scan speed may be in the range from 10 mm/s to 10000 mm/s, preferably 20-7000 mm/s, more preferably from 50-6000 mm/s.

According to the present invention it is preferred to use a layer height in the range of 0.001 mm to 10 mm, preferably 0.025 mm to 0.5 mm, more preferably 0.05 to 0.25 mm, such as 0.1 mm. It is believed that reduction in the layer thickness provides better i.e. increased print resolution of the object printed.

Suitable beam spot size for the present invention is typically in the range of from 0.0025 mm to 1 mm, for example 0.05-0.5 mm, preferably 0.1-0.3 mm, for example 0.2 mm. Increasing the spot size can be used to increase the laser beam interaction time. Typically, this is influenced by adjusting the scan speed, however.

The surface temperature is the temperature of the powder that is being sintered. Typically the surface temperature will be in the range of 0-200 degrees centigrade, preferably 40-180 degrees centigrade, most preferably 70-170 degrees centigrade The chamber temperature is the temperature within the chamber in which printing is taking place. Typically this is in the range of 20-200 degrees centigrade, preferably 20-50 degrees centigrade for low-melting polymers, e.g. poloxamers or PEGs or preferably 50-200 degrees centigrade, more preferably 60-150 degrees centigrade for high-melting polymers, e.g. PVAs.

The first and second sinter powder are loaded into the printer and the printing process is initiated. In the printing process, parameters such as chamber and print bed temperature are set to appropriate values obtained via experimental studies to provide printed pharmaceutical dosage forms with desirable properties with respect to mechanical and morphological properties. Other parameters influencing the process are laser energy input and layer height of each applied layer. The laser energy input can be controlled in a number of ways depending on which type of printer is used and usually via adjusting laser scanning speed, hatching space (distance between scanned laser lines) or by adjusting the energy output by the laser.

Once the printing process is finalized the printed pharmaceutical dosage forms are allowed to slowly cool down in the printer before being removed and cleaned from surrounding, unsintered powder.

According to the invention, the term “active pharmaceutical ingredient” or “API” is a biologically active agent or neutraceutic, preferably a biologically active agent. The API may be a small molecule in form of a weak base, a weak acid or a neutral molecule and may be in the form of one or more pharmaceutically acceptable salts, esters, derivatives, analogues, prodrugs, and solvates thereof. The first sinter powder may comprise more than one API. In one embodiment the API is poorly soluble or a lipophilic API.

As used herein, the terms “poorly soluble API”, “poorly water-soluble API” and “lipophilic API” refer to an API having a solubility such that the highest therapeutic dose of the particular API to be administered to an individual cannot be dissolved in 250 ml of aqueous media ranging in pH from 1 to 8 following the definition of low solubility according to the Biopharmaceutics Classification System (BCS) classes 2 and 4. Poorly soluble APIs with weakly basic or weakly acidic characteristics have a pH-dependent solubility profile and can have a wide range of solubility in the aqueous environment of the gastrointestinal tract. APIs falling under BCS classes 2 or 4, respectively, are well known to persons skilled in the art.

In one embodiment the API is a weakly basic API. As used herein, the term “weakly basic API” refers to a basic active pharmaceutical ingredient (API) wherein the basic API does not completely ionize in water.

The at least one active pharmaceutical ingredient (API) according to the invention may be dispersed in the pharmaceutical dosage form, forming an amorphous solid dispersion.

As used herein, the term “amorphous solid dispersion” is a dispersion of at least one amorphous API in an excipient matrix of the pharmaceutical dosage form. Preferably, the amorphous API is distributed in a molecularly dispersed state within the excipient matrix. In this case, the solid dispersion is a solid solution. Upon dissolution, formulations comprising an amorphous solid dispersion can reach higher solubilities in aqueous media than the crystalline API.

The API included in the pharmaceutical dosage form of the present invention has a sufficient amount to be therapeutically effective. For a given API, therapeutically effective amounts are generally known or readily accessible by persons skilled in the art. Typically, the API may be present in the pharmaceutical dosage form in a weight ratio of API to excipient of 0.1:99.1 to 60:40, preferably 1:99 to 50:50, more preferably 5:95 to 40:60 and most preferably 10:90 to 30:70.

According to the invention, the term “excipient” has its standard meaning in the art, i.e. a substance formulated alongside the active pharmaceutical ingredient of a medication, included for the purpose of long-term stabilization, bulking up solid formulations that contain potent active ingredients, or to confer a therapeutic enhancement on the active ingredient in the final dosage form, such as facilitating drug absorption, reducing viscosity, or enhancing solubility.

In a preferred embodiment, the excipient absorbs electromagnetic radiation at a wavelength emitted by the laser. Said electromagnetic radiation is electromagnetic radiation within the infrared, visible or ultraviolet regions of the electromagnetic spectrum.

In a preferred embodiment, applicable to all aspects of the invention, the excipient comprises or consists of a polymer.

A wide range of polymer excipients used to manufacture solid pharmaceutical dosage forms exists. These include, for example, acrylic-derived polymers, cellulose-derived polymers and polyvinyl-derived polymers and mixtures thereof. The terms “polymer” and “polymeric materials” are used herein interchangeably. Examples of suitable polymers are methyl acrylate-methacrylic acid copolymers, ethyl acrylate-methacrylic acid copolymers, cellulose acetate phthalate (CAP), cellulose acetate succinate, hydroxy propyl methyl cellulose phthalate, hydroxypropylmethylcellulose acetate succinate (HPMC-AS), polyvinyl acetate phthalate (PVAP), methyl methacrylate-methacrylic acid copolymers, shellac, cellulose acetate trimellitate, sodium alginate, zein, polyethylene oxide, ethylcellulose, hydroxypropyl cellulose, hydroxypropyl methylcellulose, polyvinyl alcohol (PVA), polyvinylpyrrolidone (PVP), vinylpyrrolidone-vinyl acetate copolymers, gelatin, polysaccharides and mixtures thereof.

In a preferred embodiment, the polymer is polyvinyl alcohol (PVA).

Polyvinyl alcohol (PVA) is a synthetic water-soluble polymer that has the idealized formula [CH₂CH(OH)]ₙ. It possesses good film-forming, adhesive, and emulsifying properties. PVA is prepared from polyvinyl acetate, where the functional acetate groups are either partially or completely hydrolysed to alcohol functional groups. If not completely hydrolysed, PVA is a random copolymer consisting of vinyl alcohol repeat units —[CH₂CH(OH)]— and vinyl acetate repeat units —[CH₂CH(OOCCH₃)]—. The polarity of PVA is closely linked to its molecular structure. The hydrolysis degree and the molecular weight determine the molecular properties of PVA. As the degree of hydrolysis of acetate groups increases, the solubility of the polymer in aqueous media and also crystallinity and melting temperature of the polymer increase. However, at high hydrolysis degrees over 88%, the solubility of PVA decreases again. PVA is generally soluble in water, but almost insoluble in almost all organic solvents, excluding, in some cases, ethanol.

The typical PVA nomenclature indicates the viscosity of a 4% solution at 20° C. and the degree of hydrolysis of the polymer. For example, PVA 4-88 is a PVA grade with a viscosity of 4 mPas that is 88% hydrolysed, i.e. having 88% of vinyl alcohol repeat units and 12% of vinyl acetate repeat units. A skilled person is aware that a hydrolysis grade of 88% and a viscosity of 4 mPas encompasses calculated hydrolysis grades of 87.50% to 88.49% and calculated viscosities of 3.50 mPas to 4.49 mPas according to common rounding methods. Viscosity according to the invention is measured as stated in USP 39 under Monograph “Polyvinyl Alcohol” with the method Viscosity-Rotational Method (912).

The degree of hydrolysis according to the invention is measured by determining the saponification value of the Polyvinyl Alcohol, e.g. as stated in USP 39 under Monograph “Polyvinyl Alcohol” under “Degree of Hydrolysis”:
Sample: 1 g of Polyvinyl Alcohol, previously dried at 110° to constant weight

### Analysis:

Transfer the Sample to a wide-mouth, 250-ml conical flask fitted by means of a suitable glass joint to a reflux condenser. Add 35 ml of dilute methanol (3 in 5), and mix gently to ensure complete wetting of the solid. Add 3 drops of phenolphthalein TS, and add 0.2 N hydrochloric acid or 0.2 N sodium hydroxide if necessary, to neutralize. Add 25.0 ml of 0.2 N sodium hydroxide VS, and reflux gently on a hot plate for 1 h. Wash the condenser with 10 ml of water, collecting the washings in the flask, cool, and titrate with 0.2 N hydrochloric acid VS. Concomitantly perform a blank determination in the same manner, using the same quantity of 0.2 N sodium hydroxide VS.
Calculation of saponification value:
Calculate the saponification value:

$$Result = [ ( {V}_{B} - {V}_{S} ) \times N \times {M}_{r} ] / W$$

V_{B}=volume of 0.2 N hydrochloric acid VS consumed in the titration of the blank (ml)
V_{S}=volume of 0.2 N hydrochloric acid V_{S} consumed in the titration of the Sample solution (ml)
N=actual normality of hydrochloric acid V_{S}
Mᵣ=molecular weight of potassium hydroxide, 56.11
W=weight of the portion of Polyvinyl Alcohol taken (g)

### Calculation of Degree of Hydrolysis:

Calculate the degree of hydrolysis, expressed as a percentage of hydrolysis of polyvinyl acetate:

$$Result = 100 - [ 7.84 \times S / ( 100 - 0.075 \times S ) )$$

S=saponification value of the Polyvinyl Alcohol

The use of PVA grades according to the invention is of interest for the formulation of solid oral pharmaceutical dosage forms with an instant, immediate or prolonged API release.

Preferred PVAs have a hydrolysis degree of 70% to 90%, and a viscosity of a 4% solution at 20° C. of 3 mPas to 8 mPas, more preferably a viscosity of a 4% solution at 20° C. of 3 mPas to 5 mPas, most preferably a viscosity of a 4% solution at 20° C. of 3 mPas to 4 mPas.

In a further embodiment of the invention, the polyvinyl alcohol has a hydrolysis degree of 70% to 90%, preferably 80% to 90% and a viscosity as mentioned above.

In a further embodiment of the invention, the polyvinyl alcohol has a hydrolysis degree of 80% to 90% and a viscosity of a 4% solution at 20° C. of 3 mPas or a hydrolysis degree of 80% to 90% and a viscosity of a 4% solution at 20° C. of 4 mPas.

In a further embodiment of the invention, the polyvinyl alcohol is PVA 3-80, PVA 3-82, PVA 4-88, PVA 5-88, PVA 8-88 or PVA 5-74, preferably PVA 3-80, PVA 3-82 or PVA 4-88, more preferably PVA 4-88.

In a further embodiment of the invention, the polyvinyl alcohol is PVA 3-82, PVA 4-88 or PVA 5-74, more preferably PVA 3-82 or PVA 4-88, most preferably PVA 3-82.

In a further preferred embodiment, the polymer is a poloxamer (PLX).

Poloxamers are amphiphilic polymers, with two hydrophilic blocks and a hydrophobic block in the middle. A poloxamer is a polyethylene glycol (PEG)/polypropylene glycol (PPG) tri-block copolymer whereby one PPG block is flanked on both sides with a PEG block. The polyethylene glycol (PEG) part is often also called polyethylene oxide (PEO) part. The polypropylene glycol (PPG) part is often also called the polypropylene oxide (PPO) part.

Poloxamer grades are commonly named with the letter P (for poloxamer) followed by three digits that is officially used by USP and EP. It describes the composition of the polymer as follows: the first two digits multiplied by 100 represents the molecular weight of the PO block and the last digit multiplied by 10 provides the percentage of EO in %.

Poloxamer P188 in average is composed of 80% EO, while the remaining 20% PO make up for 1800 g/mol. Poloxamer P407 is a poloxamer with an average polyoxypropylene molecular mass of 4000 g/mol and a 70% polyoxyethylene content.

Poloxamers have the general formula (I)

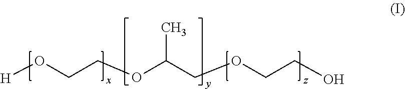

For different poloxamers numbers of x (PEO), y (PPO chain) and z (PEO) are varying over a broad range, depending on the type of poloxamer. For poloxamer P188 the PPO chain contains in the average a unit number ranging from 25 to 30, and each PEO is composed of 75 to 85 EO units in average, with a molecular weight ranging from 7680 to 9510 Da. For poloxamer P407 the PPO chain contains in the average a unit number of 56, and each PEO is composed of approximately 101 EO units in average, with a molecular weight ranging from 9840 to 14600 Da.

Poloxamer 407 (a=101, b=56) with molecular weight ranging from 9840 to 14600 Da.

Table 1 is showing types of poloxamers monographed in the European Pharmacopoeia (Ph. Eur.) and United States Pharmacopeia (USP).

TABLE 1

| | Average | | EO units | |
|---|---|---|---|---|
| | Molecular | | (each flank | PO units Ph. |
| | Weight | Weight EO | Ph. Eur. | Eur. (range); |
| Poloxamer | (USP/EP) | [%] USP | (range); USP | USP |
| 124 | 2090-2360 | 46.7 ± 1.9 | 10-15; 12 | 18-23; 20 |
| 188 | 7680-9510 | 81.8 ± 1.9 | 75-85; 80 | 25-30; 27 |
| 237 | 6840-8830 | 72.4 ± 1.9 | 60-68; 64 | 35-40; 37 |
| 338 | 12,700-17,400 | 83.1 ± 1.7 | 137-146; 141 | 42-47; 44 |
| 407 | 9840-14,600 | 73.2 ± 1.7 | 95-105; 101 | 54-60; 56 |

Preferred poloxamers are P188 and P407.

In a further embodiment of the invention, the second sinter powder may comprise further pharmaceutically acceptable additives. Pharmaceutically acceptable additives comprise flow control agents, such as silicon dioxide, fillers, plasticizers, surfactants, light-absorbing material, such as ruby red or Candurin pigments and other suitable components that are well known to those skilled in the art.

In one embodiment of the invention, the second sinter powder further comprises a light-absorbing material.

Depending on the wavelength of the light emitted by the laser, a light-absorbing material (pigment) which absorbs light at the wavelength emitted may be required. These light-absorbing materials can contain transition metals for absorption at around 450 nm or carbon for a wider range covering the visible—and near IR range. Light absorption is a process by which light is absorbed and converted into energy. When light is absorbed heat is generated. So the selective absorption of light by a particular material occurs because the frequency of the light wave matches the frequency at which electrons in the atoms of that material vibrate.

Light-absorbing materials are all materials suitable for the SLS method as described above and known to the skilled person in the art. Preferably light-absorbing materials which have been demonstrated to work at 455 nm laser irradiation are used, e.g. Candurin NXT, Ruby Red, Candurin Gold Sheen, Aluminum Lake, activated carbon (also works at 808 nm) or iron oxide (Fe₂O₃). More preferably ruby red is used.

Carbon dioxide laser emitting at around 9-10 microns will usually not require addition of light-absorbing materials as C—H bonds will absorb energy at this wavelength and this type of bond can be found in most polymers.

For the avoidance of doubt, further pharmaceutically acceptable additives as defined above are not needed for the beneficial properties according to the invention. Yet those additives can be used for other purposes, e.g. to optimize the process of manufacturing of the pharmaceutical composition or oral dosage form according to the invention.

Furthermore, the pharmaceutical composition according to the invention may comprise additional pharmaceutically acceptable hydrophilic or lipophilic polymers.

As used herein, the phrase “pharmaceutically acceptable” refers to all excipients, additives, polymers, compounds, solvents, dispersion media, flow control agents, carriers, coatings, active agents, isotonic and absorption delaying agents, and the like that do not produce an allergic or similar untoward reaction when administered to humans in general. The use of such material in pharmaceutical compositions is well known in the art.

In a preferred embodiment the particle size of the first and second sinter powder has a D50 of 200 μm or lower. Preferably, the particle size (D50) of the sinter powder is between 20 μm and 200 μm, 20 μm and 150 μm or 20 μm and 100 μm.

In a preferred embodiment the particle size of the PVA has a D50 of 200 μm or lower. Preferably, the particle size (D50) of the PVA is between 20 μm and 200 μm, 20 μm and 150 μm or 20 μm and 100 μm.

In a further preferred embodiment the particle size of the PVA has a D90 of 250 μm or lower. Preferably, the particle size (D90) of the PVA is between 100 μm and 250 μm, more preferably between 140 μm and 220 μm.

A further embodiment of the invention is a pharmaceutical dosage form obtainable by the process as described above.

According to the invention, a “pharmaceutical dosage form” means a dosage form or unit dose containing a drug for pharmaceutical application. Typically the dosage forms may be cylindrical, spherical, prismatic, oval, capsule-shape, or elongate, or diamond shaped. The pharmaceutical dosage forms may be prepared with a variety of physical forms, including tablets, caplets, orally-disintegrating tablets, films, masks and patches. The pharmaceutical dosage forms may be prepared with a range of release-behaviour. The pharmaceutical dosage forms according to the invention may provide at least one of modified release, immediate release, colonic delivery, enteric delivery, or gastroretentive drug delivery performance. Preferably, immediate release or controlled-release is provided.

The pharmaceutical dosage forms may be suitable for administration via oral, buccal, topical, transdermal, sublingual, enteral, dental, rectal, urethral or vaginal routes. Preferably oral, buccal, topical or transdermal administration may be used, more preferable oral administration is used.

It was surprisingly found that the pharmaceutical dosage forms printed with the multi-compartmental method have an improved structure consistency. The tablets have a more accurate geometry, e.g. they do not have a curvature at the bottom of the structure as compared to pharmaceutical dosage forms printed with the conventional method.

Furthermore, improved surface quality and parameters can be measured by scanning electron microscope (SEM) method, BET (gas adsorption) or uCT measurements.

Additionally, pharmaceutical dosage forms printed with the multi-compartmental method show a faster and higher API release and/or an improved amorphization of the API compared to pharmaceutical dosage forms printed with the conventional method. The API release can be tested by dissolution experiments in aqueous media. Improved amorphization of pharmaceutical dosage forms manufactured by the multi-compartmental method can be seen when Powder X-ray diffraction (PXRD) data and/or differential scanning calorimetry (DSC) data are compared to pharmaceutical dosage forms manufactured by the conventional method.

Furthermore, with the multi-compartmental method, the drug content of the individual dosage forms can be adjusted within the same printing step or between different printing step with the identical powder mixtures, which can be realized by adapting the amount of first and second sinter powder. This adaption is not possible with the conventional method as described above.

In addition, de-mixing between API and excipient can happen with the conventional method which leads to a poor content uniformity. This effect is less likely to be observed with the multi-compartmental method.

### EXAMPLES

### Example 1: SLS Printing with Conventional Method

### Formulation Preparation

The formulation for the conventional SLS 3D printing is a blend of the PVA-based polymer (PVA), active pharmaceutical ingredient (API) and the flowability improver (silicon dioxide colloidal (SD)). The detailed information on the composition is provided in the table 2 below.

TABLE 2
Composition of the formulation for conventional method

| Component | Trade name | Manufacturer | [wt %] |
|---|---|---|---|
| PVA | Parteck ® MXP, | Merck KGaA | 49.25 |
| | PVA 3-82 | | |
| SD | colloidal, highly | Merck KGaA | 1.5 |
| | dispersed, EMPROVE ® | | |
| | ESSENTIAL | | |
| API | Indomethacin | Tokyo Chemical | 49.25 |
| | | Industry Co., LTD | |

The following formulation preparation sequence was used:
1. The PVA-based polymer, SD, and API were manually weighted and mixed together.
2. The blend was sieved using 315 μm stainless-steel test sieve (VWR International AB, Stockholm, Sweden). This step is needed to get rid of the SD lumps and bigger particle agglomerates of API and PVA-based polymer.
3. Then, the sieved blend was mixed for 3 hours using a Turbula shaker (Turbula T2F shaker, Glen Mills, Inc., Clifton, NJ, US).
4. The mixed blend was heat treated at 70° C. in order to exclude the Temperature Memory Effect (TME) of the polymer and water residuals.
5. Then, the blend was mixed again for 2 hours using a Turbula shaker.
6. The final formulation was loaded into the powder compartments of the printer.

### Hardware Setup and SLS Printing Procedure

The SNOWWHITE2 Selective Laser Sintering 3D printer (SHAREBOT S.R.L., Nibionno, Lecco, Italy) is used as a work system. The printer implements sintering via CO₂ (λ=10.6 μm, P=14 W) galvanometer laser system. The printer has an F-Theta lens in order to avoid aberrations such as field curvature. The space inside of the printer is split into three compartments: left, right, and building compartment. Left and right compartments are used for the powder supply, whereas the actual sintering occurs on the surface of the building compartment.

### SLS Printing Procedure:

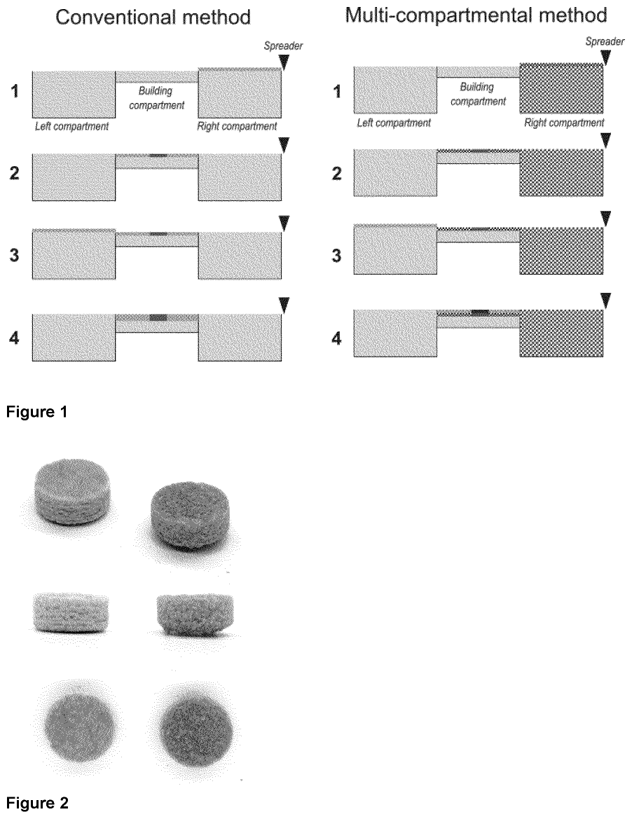

1. Both right and left powder supply compartments are filled with the same formulation in case of the conventional method.
2. The heating lamps warm up the powder inside of the printer to the set temperature.
3. Five “warm-up” powder layers are applied into building compartment from the left and right compartments in rotation. It is needed to avoid temperature gradient inside of the building compartment. The spreader (black triangle in FIG. 1) is used for the powder layer applications. The layer application process sequence is the same during the whole printing process:
   a) The left or right powder supply compartment moves up and creates the excessive layer of powder (step 1 in FIG. 1).
   b) The central building compartment moves down (the distance is equal to the structure layer height) creating the empty space for the structure layer.
   c) The spreader moves along the powder surface drugging the excessive powder layer into the building compartment. This powder fills the empty space in the building compartment.
4. Then, the sintering occurs upon the powder surface in the building chamber (step 2 in FIG. 1).
5. After that the sequence repeats for the opposite compartment in rotation each new layer.
6. Then, the whole printer chamber is cooled down and printer structure can be picked up.

### Printing Settings

The printing setting is shown in the table 3 below.

TABLE 3
Printing settings for conventional method

| | Conven- | |
|---|---|---|
| Parameter | tional | Comments |
| Rate, pps | 60000 | Laser movement speed. Pps - points |
| | | per second. 1 pps = 0.06 mm |
| Power, % | 30 | Percentage of the initial (14 W) laser |
| | | power |
| Border rate, pps | 60000 | Laser movement speed for the outer |
| | | part of the object |
| Border power, % | 30 | Same as the power but for the outer |
| | | part of the object |
| Temperature | Powder | The temperature control based on the |
| control | | powder temperature in the building |
| | | compartment |
| Plate temperature, | 130 | |
| ° C. | | |
| Layer height, μm | 125 | This value defines the distance the |
| | | building plate moves at |
| Warming layer | 5 | |
| Powder layer, mm | 0.2 | The distance powder supply |
| | | compartments move at during |
| | | warming-up step |
| Recoater speed, | 9000 | How fast the spreader moves along |
| mm/min | | the powder surface |
| Change at every | — | The powder supply chambers |
| | | switched each 2 layers |
| Start | — | The starting powder supply chamber. |
| | | Also used for the warming-up step |
| | | (all 5 warming layers are applied from |
| | | the left compartment) |
| Powder layer left, | — | The distance polymer compartment |
| mm | | moves at during the printing step |
| Powder layer right, | — | The distance API compartment |
| mm | | moves at during the printing step |

### Example 2: SLS Printing with Multi-Compartmental Method

### Formulation Preparation

The multi-compartmental method assumes using formulation excipients substances without the prior mixing. Each compartment is filled with the different excipient. The API and the polymer were used as the compartment fillers. The SD was added to each excipient in order to keep the same flowability properties. The detailed information on the composition is provided in table 4 below.

TABLE 4
Composition of the formulations for multi-compartmental method

| Compartment | Component | Trade name | Manufacturer | [wt %] |
|---|---|---|---|---|
| Left | PVA | Parteck ® MXP, | Merck KGaA | 98.5 |
| | | PVA 3-82 | | |
| | SD | colloidal, highly | Merck KgaA | 1.5 |
| | | dispersed, EMPROVE ® | | |
| | | ESSENTIAL | | |
| Right | API | Indomethacin | Tokyo | 98.5 |
| | | | Chemical | |
| | | | Industry Co., | |
| | | | LTD | |
| | SD | colloidal, highly | Merck KgaA | 1.5 |
| | | dispersed, EMPROVE ® | | |
| | | ESSENTIAL | | |

The following formulation preparation sequence was used:

### Left Compartment Formulation (PVA)

1. The PVA-based polymer was manually weighted and mixed with SD.
2. The blend was sieved using 315 μm stainless-steel test sieve (VWR International AB, Stockholm, Sweden). This step is needed to get rid of the SD lumps and bigger particle agglomerates of PVA-based polymer
3. Then, the sieved blend was mixed for 3 hours using a Turbula shaker (Turbula T2F shaker, Glen Mills, Inc., Clifton, NJ, US).
4. The mixed blend was heat treated at 70° C. in order to exclude the Temperature Memory Effect (TME) of the polymer and water residuals.
5. Then, the blend was mixed again for 2 hours using a Turbula shaker.
6. The final formulation was loaded into the left and central compartment

### Right Compartment Formulation (API)

1. The API was manually mixed weighted and with SD.
2. The blend was sieved using 315 μm stainless-steel test sieve (VWR International AB, Stockholm, Sweden). This step is needed to get rid of the SD lumps and bigger particle agglomerates of API.
3. Then, the sieved blend was mixed for 3 hours using a Turbula shaker (Turbula T2F shaker, Glen Mills, Inc., Clifton, NJ, US).
4. The final formulation was loaded into the right compartment

### Hardware Setup and SLS Printing Procedure

The SNOWWHITE2 Selective Laser Sintering 3D printer (SHAREBOT S.R.L., Nibionno, Lecco, Italy) is used as a work system. The printer implements sintering via CO₂ (λ=10.6 μm, P=14 W) galvanometer laser system. The printer has an F-Theta lens in order to avoid aberrations such as field curvature. The space inside of the printer is split into three compartments: left, right, and building compartment. Left and right compartments are used for the powder supply, whereas the actual sintering occurs on the surface of the building compartment.

### SLS Printing Procedure:

1. Both left and building compartments are filled with a PVA-based polymer powder, whereas the right is filled with API powder. Different powders have different infill in FIG. 1.
2. The heating lamps warm up the powder inside of the printer to the set temperature.
3. Five “warm-up” powder layers are applied into building compartment from the left compartment only. It is needed to avoid temperature gradient inside of the building compartment. The spreader (black triangle in FIG. 1) is used for the powder layer applications. The layer application process sequence is the same during the whole printing process:
   a) The left or right powder supply compartment moves up and creates the excessive layer of powder (step 1 in FIG. 1).
   b) The central building compartment moves down (the distance is equal to the structure layer height) creating the empty space for the structure layer.
   c) The spreader moves along the powder surface drugging the excessive powder layer into the building compartment. This powder fills the empty space in the building compartment.
4. The sintering occurs upon the powder surface in the building chamber (step 2 in FIG. 1).
5. After that the sequence repeats for the opposite compartment in rotation each 2 new layer.
6. Then, the whole printer chamber is cooled down and printer structure can be picked up.

### Printing Settings

The printing setting is shown in the table 5 below.

TABLE 5
Printing settings for multi-compartmental method

| | Multi- | |
|---|---|---|
| Parameter | compartmental | Comments |
| Rate, pps | 85000 | Laser movement speed. Pps - points |
| | | per second. 1 pps = 0.06 mm |
| Power, % | 26 | Percentage of the initial (14 W) laser |
| | | power |
| Border rate, | 85000 | Laser movement speed for the outer |
| pps | | part of the object |
| Border power, | 26 | Same as the power but for the outer |
| % | | part of the object |
| Temperature | Powder | The temperature control based on the |
| control | | powder temperature in the building |
| | | compartment |
| Plate | 125 | |
| temperature, | | |
| ° C. | | |
| Layer height, | 125 | This value defines the distance the |
| μm | | building plate moves at |
| Warming layer | 5 | |
| Powder layer, | 0.2 | The distance powder supply |
| mm | | compartments move at during |
| | | warming-up step |
| Recoater | 9000 | How fast the spreader moves along the |
| speed, mm/min | | powder surface |
| Change at | 2 | The powder supply chambers switched |
| every | | each 2 layers |
| Start | Left | The starting powder supply chamber. |
| | | Also used for the warming-up step (all |
| | | 5 warming layers are applied from the |
| | | left compartment) |
| Powder layer | 0.3 | The distance polymer compartment |
| left, mm | | moves at during the printing step |
| Powder layer | 0.3 | The distance API compartment moves |
| right, mm | | at during the printing step |

Even though both methods, the conventional and multi-compartmental method, use the same substances for formulation preparation, the optical and thermal properties of the blend and pure materials are different. Therefore, the conventional and the multi-compartmental method could not be performed with the same printing setting and printing settings were adjusted to avoid oversintering and powder layers wrapping because of the temperature gradient. In the following, details for the selection of differences in rate, powder and plate temperature parameters between the conventional and multi-compartmental method are presented:
The higher rate (85 000 pps) and lower energy (26%) were selected because of the pure polymer wrapping issue. These two values define how much laser energy would be transferred upon the building chamber powder surface. In case of the blend formulation energy is distributed between the polymer and API which needs more energy than polymer and API separately.
The plate temperature decrees in case of multi-compartmental method is caused by the melting of the top layer of API in the right compartment. It is not an issue for the blend because of the polymer presence which needs higher temperature to be molten.

### Example 3: Characterization of 3D Printed Dosage Forms

### Structure Consistency

The printing model is the cylinder with 10 mm diameter and 4 mm height. The total number of layers is 32 (4/0.125=32, where 4 is height (mm), 0.125 is a layer height (mm)) for both methods. All layers consist of the blend formulation in case of the conventional method. In case of the multi-compartmental method, 16 layers are polymer-based and 16 are API-based. API and polymer layers alternate each two layers. The final structures printed by both methods in FIG. 2 shows the final structure of the tablets printed by the conventional method (right side) and the multi-compartmental method (left side). It can be seen that tablets printed using the conventional method are darker because of the higher energy transfer and consequently more intense API and polymer melting. However, the multi-compartmental method demonstrates better structure consistency, e.g. those tablets does not show a curvature at the bottom of the structure as compared to tablets printed with the conventional method.

1. Process for producing a pharmaceutical dosage form by powder bed fusion selective laser 3-dimensional printing, comprising the steps of
(a) providing a first sinter powder, essentially consisting of an active pharmaceutical ingredient or essentially consisting of a mixture of at least two active pharmaceutical ingredients, in a first reservoir platform,
(b) providing a second sinter powder, comprising an excipient, wherein said excipient absorbs electromagnetic radiation at a wavelength emitted by the laser, in a second reservoir platform, and
(c) operating a selective laser sintering apparatus that fuses layers of the first and second sinter powder to produce the pharmaceutical dosage form.

2. Process according to claim 1, wherein the laser is a CO₂ laser.

3. Process according to claim 1, wherein the second sinter powder further comprises at least one light-absorbing material.

4. Process according to claim 1, wherein said powder bed fusion selective laser 3-dimensional printing comprises selective laser sintering 3-dimensional printing, selective laser melting 3-dimensional printing, electron beam melting 3-dimensional printing or multijet fusion or a mixture thereof.

5. Process according to claim 1, wherein said powder bed fusion selective laser 3-dimensional printing is selective laser sintering 3-dimensional printing.

6. Process according to claim 1, wherein said excipient comprises of a polymer.

7. Process according to claim 6, wherein said polymer is selected from the group consisting of acrylic-derived polymers, cellulose-derived polymers, poloxamers and polyvinyl-derived polymers and mixtures thereof.

8. Process according to claim 6, wherein said polymer is selected from the group consisting of methyl acrylate-methacrylic acid copolymers, ethyl acrylate-methacrylic acid copolymers, cellulose acetate succinate, hydroxy propyl methyl cellulose phthalate, hydroxypropyl methyl cellulose acetate succinate, polyvinyl acetate phthalate, methyl methacrylate-methacrylic acid copolymers, shellac, cellulose acetate trimellitate, sodium alginate, zein, polyethylene oxide, ethylcellulose, hydroxypropyl cellulose, hydroxypropyl methylcellulose, polyvinyl alcohol, poloxamers, polyvinylpyrrolidone, vinylpyrrolidone-vinyl acetate copolymers, gelatin, polysaccharides and mixtures thereof.

9. Process according to claim 6, wherein said polymer is a polyvinyl alcohol.

10. Process according to claim 6, wherein said polymer is a polyvinyl alcohol having a hydrolysis degree of 70% to 90%, and a viscosity of a 4% solution at 20° C. of 3 mPas to 8 mPas.

11. Process according to claim 6, wherein said polymer is polyvinyl alcohol PVA 4-88 or PVA 3-82.

12. Process according to claim 1, wherein said electromagnetic radiation is electromagnetic radiation within the infrared, visible or ultraviolet regions of the electromagnetic spectrum.

13. Pharmaceutical dosage form produced by the process of claim 1.

14. Pharmaceutical dosage form according to claim 13, wherein the pharmaceutical dosage form is an oral dosage formulation.
